(19) United States
(12) Patent Application Publication
(10) Pub. No.: US 2006/0264760 A1
(43) Pub. Date: Nov. 23, 2006

(54) Near infrared transrectal probes for prostate cancer detection and prognosis

(75) Inventors: Hanli Liu, Arlington, TX (US); Thomas C. Truelson, Fort Worth, TX (US)

Correspondence Address: GARDERE WYNNE SEWELL LLP;INTELLECTUAL PROPERTY SECTION, 3000 THANKSGIVING TOWER, 1601 ELM ST, DALLAS, TX, 75201-4761, US

(73) Assignee: Board of Regents, The University of Texas System, Austin, TX (US)

(21) Appl. No.: 11/352,018

(22) Filed: Feb. 10, 2006

Related U.S. Application Data
(60) Provisional application No. 60/651,825, filed on Feb. 10, 2005.

(51) Int. Cl. A61B 6/00

(52) U.S. Cl. 600/473

(57) ABSTRACT

A multi-channel, near infrared (NIR) imaging system comprising one or more optical fiber bundles; and a transrectal NIR probe having an outer material, wherein the optical fiber bundle is integrated into the transrectal probe (an end-fire or a side-fire NIR probe). The outer material is preferably rubber-like silicon or made of two translucent materials exhibiting low viscosity and requires curing at room temperature. The transrectal NIR probe comprises a curvature to match the curvature of a human rectum and may have a circular or oval-shaped silicone holder. The imaging system further comprises a broad-band light source adapted to deliver light to the fiber channels, an optical switch box adapted to allow only one of the fiber channels to pass through at a time; and a multi-channel spectrometer to capture tomographic images of changes in HbO, HbT, light scattering patterns and hemodynamic response times from human prostate.

### BACKGROUND

The present invention relates to the general field of cancer detection, and in particular, to transrectal probes for prostate cancer detection and prognosis.

Prostate cancer is the most common male cancer and the second leading cause of cancer death in men in the United States. Effective treatments are highly associated with accurate and early detection of prostate cancer. It is currently difficult, however, to detect early stages of prostate cancer because of lack of any obvious symptoms. There are four common examination and screening methods known in the art for screening prostate cancer: Digital Rectal Examination (DRE), Transrectal Ultrasonography (TRUS), Prostate-Specific Antigen (PSA) test and Magnetic Resonance Imaging (MRI) and Magnetic Resonance Spectroscopic Imaging (MRSI). All of these early detection and screening tests have serious shortcomings as discussed below.

DRE is the most established method of examining the prostate gland. The clinician inserts a lubricated index finger into the rectum and palpates the posterior Surface of the prostate gland. This examination allows a rough assessment of the shape, configuration, symmetry and consistency of the prostate. The clinician may also estimate the prostate size, but because only the posterior aspect can be palpated, the estimate is usually fairly inaccurate. Although the clinician may feel indurations and nodules, the sensitivity and specificity for detecting prostate cancer is very low. DRE is, thus, an ineffective prostate cancer screening tool.

The second method of detection known in the art, TRUS, is an imaging modality utilizing ultrasound of a frequency between 6 and 8 MHz. TRUS obtains relatively high resolution images of the prostate, usually in 64 shades of gray. Ultrasound emitting crystals are usually mounted on a transducer less than one inch in diameter. The transducer is inserted into the rectum and brought in direct proximity of the prostate gland. The focal point and range of the transducer is chosen to allow imaging in transverse and longitudinal orientation with a depth of field up to 5-10 cm, thus allowing imaging of even large prostate glands. There are two basic ultrasound probes used in TRUS, namely end-fire and side-fire probes, each differing in the way the ultrasound emitting crystal is mounted onto the transducer head. FIG. 1, for example, is an illustrative depiction of TRUS with a multi-plane, side-fire probe. Sophisticated image analysis software, as well as artificial neural networks (ANN), further improved predictive ability, but TRUS has remained a research tool and has had very little impact on clinical practice. TRUS does serve, however, allows a physician to perform image guided needle biopsies of the prostate either by aiming randomly at various zones of the prostate or specifically at areas of suspicion. It has been shown, however, that areas of the prostate noted to be more hypoechoic (in comparison to normal prostate tissue) are more likely to harbor prostate cancer, while areas more hyperechoic are less likely to be cancerous. Thus, improving the sensitivity and specificity of TRUS to predict the presence or absence of cancer has largely remained futile.

The third prostate cancer detecting method is the PSA test. PSA is the enzyme most widely used as a serum marker for prostate cancer. PSA is involved in the liquefaction of the seminal coagulum, which is present in extremely high concentrations in the seminal fluid and in measurable concentration in the serum. However, recent findings suggest considerations that make such monolithic cut-off values less attractive, for example: (1) about 25% of men with serum PSA values above 4.0 ng/ml have benign prostatic hyperplasia (BPH) only and no cancer; (2) significant numbers of men have cancer even if their PSA is below 4.0 ng/ml. PSA levels, therefore, may be elevated in men with benign prostatic conditions. On the other hand, men with cancer may have relatively low levels of serum PSA. PSA, therefore, is specific to the prostate but not for prostate cancer. Based on statistical performance characteristics, a threshold of serum PSA>4.0 ng/ml had triggered a TRUS guided prostate biopsy. Currently, however, those skilled in the art no longer definitively correlate an elevated serum PSA level with prostate cancer.

Clinicians in the art currently face considerable challenges in the diagnostic evaluation a man at risk for prostate cancer. The initial screening most likely includes DRE and serum PSA measurement. Thereafter, based on the information from the initial screening, the clinician must decide whether to proceed with a TRUS guided biopsy. The TRUS imaging itself is of little help to the clinician in deciding where to biopsy and how many cores to take. In the absence of improved serum or urine markers for prostate cancer, it is therefore imperative to develop improved imaging modalities which would allow the clinicians to better determine when and where to biopsy and how many cores to take from which areas of the prostate. Accordingly, what is needed is a more reliable and quantitative system and method for prostate cancer screening and prognosis.

Lastly, MRI techniques rely on abnormal signal intensities that result from morphologic changes within prostate to find existence and extent of cancer, but MRI does not accurately reflect the presence and spatial extent of cancer. High spatial resolution endorectal-coil T2-weighted images can show the normal peripheral zone (PZ) tissue and prostate cancer since signal intensity at cancer is decreased due to increased cell density and a loss of prostate ducts. MRI alone has a good accuracy in detecting seminal vesicle invasion (96%), but assessing the cancer spread through the prostatic capsule is harder to achieve (81% accuracy).

Magnetic resonance spectroscopic imaging (MRSI), measures the level of citrate and polyamines that are reduced or absent in cancer region, while the level of choline is elevated compared to surrounding healthy tissue. Adding MRSI to endorectal-coil MRI increased overall accuracy in prostate tumor volume measurement and detection of tumor, while measurement variability still limits consistent, quantitative tumor volume estimation, particularly for small tumors (<0.5 cm³). There are also various efforts on improving the image quality of prostate cancer using MRI and/or MRSI by changing the imaging acquisition methods, correcting image distortion by motion artifacts or applying contrast materials. However, because of the cost and non-portability, MSRI is unlikely to become a practical screening tool for prostate cancer detection.

Recent studies have shown that tumor hypoxia is a possible diagnostic and prognostic indicator that is related to the aggressiveness of a tumor, the clinical stage, and therapy outcome. Detection and evaluation of tumor oxygenation distributions and their changes accurately are critical during various stages of tumor growth. Numerous studies on tumor oxygen tension measurements have been conducted in recent years using a variety of methods, such as microelectrodes, optical reflectance, EPR or MRI. The latter two methods have advantages of making repeated measurements of pO₂ non-invasively, but lack speed and portability.

It is further known in the art that light absorption and scattering in the near infrared (NIR) range are sensitive to hemoglobin concentrations and vascular volume, but still allowing the NIR signals to penetrate through deep tissues in several centimeters. Although non-invasive, quantitative, NIR spectroscopy and imaging of tissue vascular oxygenation for the brain and the breast are known in the art, NIR imaging techniques are limited by their spatial resolutions.

NIR spectroscopy imaging modality provides both structural and functional images For example, NIR studies on the brain include non-invasive detections of brain injury/trauma, determination of cerebro-vascular hemodynamics and oxygenation and functional brain imaging in response to a variety of neurological activations. Currently, the use of functional NIR (fNIR) brain imaging has increasingly become a new imaging modality for studying neuro-hemodynamic responses to brain activation because the optical signals of the NIR techniques are non-invasively penetrate through the scalp and skull of an adult human and sensitive to changes in the concentration of oxygenated (HbO) and deoxygenated hemoglobin (Hb). While it is difficult from the NIR imaging techniques to obtain very accurate quantifications of cerebral HbO and Hb concentrations due to rigorous requirements of theory and boundary conditions, the techniques can offer relatively accurate measurements on changes of HbO and Hb. Thus, NIR imaging techniques provide quantitative changes in total cerebral blood volume (CBV, which is proportional to total hemoglobin concentration, HbT, and HbT=HbO+Hb).

Although Hb, HbO and HbT concentrations and scattering properties of tumors may be different from those of surrounding tissues, the optical contrast between cancerous and normal tissue is about 2-3 fold at most in absorption, and much less in scattering. Those in the art have focused on increasing the optical contrast between tumor and healthy surrounding tissues by using fluorescence imaging or molecular beacons to detect and diagnose cancer/tumor with improved sensitivity and specificity. Efforts on using the NIR techniques to detect and monitor prostate tumors, however, have been very limited due to the difficulty in accessing human prostates using currently known optical imaging systems and probes. Therefore, what is needed is a system for providing optical imaging systems and probes, using NIR techniques, to detect and monitor prostate tumors. What is also needed is a system for transrectal, NIR imaging and compatible probes that are minimally invasive, portable and cost-effective screening or detection tools for use in routine check-up clinics for detecting, for example, prostate cancer.

Moreover, existing imaging systems fail to obtain functional imaging of tissue metabolic and oxygenation states. For example, the most common appearance for prostate cancer, shown in sonograms, is a hypoechoic lesion (dark compared to normal tissue) in the peripheral zone. With PSA based screening and other cancer detection methods, fewer overt abnormal sonographic findings are detected at the time of transrectal ultrasound guided biopsy. There are no known biologic differences between isoechoic and hypoechoic prostate cancers because ultrasound imaging/detection rests on the structural differences between tissue types and thus is limited for metabolic and physiological imaging for tissue functions.

Furthermore, because there is limited light absorption by hemoglobin at the NIR region, NIR can penetrate through deep tissues in several centimeters, depending on the tissue types. Although prostate cancer can appear within the prostate gland at various locations, the early cancer features often start near the posterior surface of the prostate gland, about a few millimeters from the inner wall of rectum. Accordingly, what is needed are better designed NIR transrectal probes, particularly side-fire probes, which allow NIR photons to penetrate deep through the most regions of the prostate gland to obtain NIR tomographic images for physiological maps of the prostate.

What is needed therefore is a transrectal, end-fire, NIR spectroscopic probe that has multi-channel light collection and can collect optical signals from, for example, 5-10 mm depth within the human prostate in vivo. What is also needed is a transrectal, side-fire, NIR tomographic imaging probe that can be used in vivo to obtain tomographic images of HbO, Hb, HbT, as, ps, and τ from human prostate.

### SUMMARY OF THE INVENTION

The present invention combines, for example, NIR tomographic imaging techniques with uniquely designed transrectal NIR probes. The present invention obtains optical and physiological signatures of prostate cancer in human, which can be, in turn, used later as finger-prints of prostate cancer to diagnose the cancer. The present invention provides transrectal NIR imaging for prostate cancer which is capable, for example, of providing heterogeneous maps of oxygenation and metabolic parameters of prostate tumors in humans. The present invention further provides an efficient, portable, real-time and minimally invasive screening/diagnostic means for prostate cancer, which will enhance knowledge in the art on prostate tumor development in its oxygenation and metabolism. The present invention provides an innovative approach to a screening modality which provides routine, early and more accurate detection for prostate cancer. The present invention provides NIR transrectal probes which allow NIR photons to penetrate deep through the most regions of the prostate gland, and thus obtain NIR tomographic images for physiological maps of the prostate.

### BRIEF DESCRIPTION OF THE DRAWINGS

The above and further advantages of the invention may be better understood by referring to the following description in conjunction with the accompanying drawings, in which:

FIG. 1 is an illustrative schematic of TRUS with a multiplane side-fire probe of the prior art;

FIG. 2A is an illustrative schematic diagram of the multi-source, 8-detector transrectal NIR imaging system;

FIG. 2B is a preferred setup of the NIR broadband system;

FIG. 3A is a preferred transrectal ultrasound probe;

FIG. 3B is a close-up view of the preferred transrectal ultrasound probe shown in FIG. 3A;

FIG. 4 depicts two preferred black silicon samples for the outer material of a preferred transrectal NIR probe;

FIG. 5A is an illustration of a preferred NIR imaging system employed on a human;

FIG. 5B is an illustration of a cross-section of a preferred end-fire transrectal probe;

FIG. 5C is an illustration of a cross-section of a preferred side-fire transrectal probe;

FIG. 6A is a depiction of the theoretical plots of normalized reflectance as a function of hemoglobin oxygen saturation;

FIG. 6B is a depiction of the comparison of normalized reflectance obtained from the experimental data and theoretical calculation;

FIG. 7A is an illustrative schematic diagram for the hemodynamic phantom;

FIG. 7B is a preferred setup for the dynamic phantom with multi-tubes imbedded; and

FIG. 8 is an illustrative flow diagram for the reconstructive algorithm.

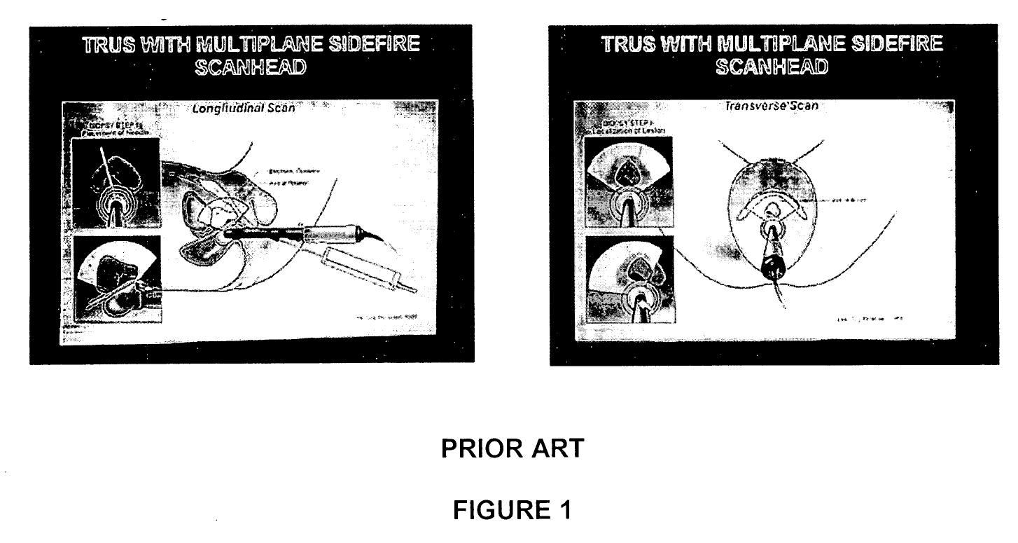

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

The present invention provides a broadband, multi-channel, tomographic NIR imaging system 10, particularly designed for the prostate cancer studies. While it is simpler to use just 2-4 wavelengths to obtain hemodynamic parameters, the present invention derives accurate concentrations of several intrinsic chromophores presented in tissue. A system in accordance with the present invention, therefore, can be used to efficiently separate both absorption and reduced scattering coefficients without any non-uniqueness problem. Thus, the present invention uniquely separates both the absorption and scattering effects, while still utilizing a CW system with the best penetration depth and best signal-to-noise ratio. Based on the principles and the advantages of NIR tomography, NIR imaging in accordance with the present invention provides a detection technique for prostate cancer diagnosis.

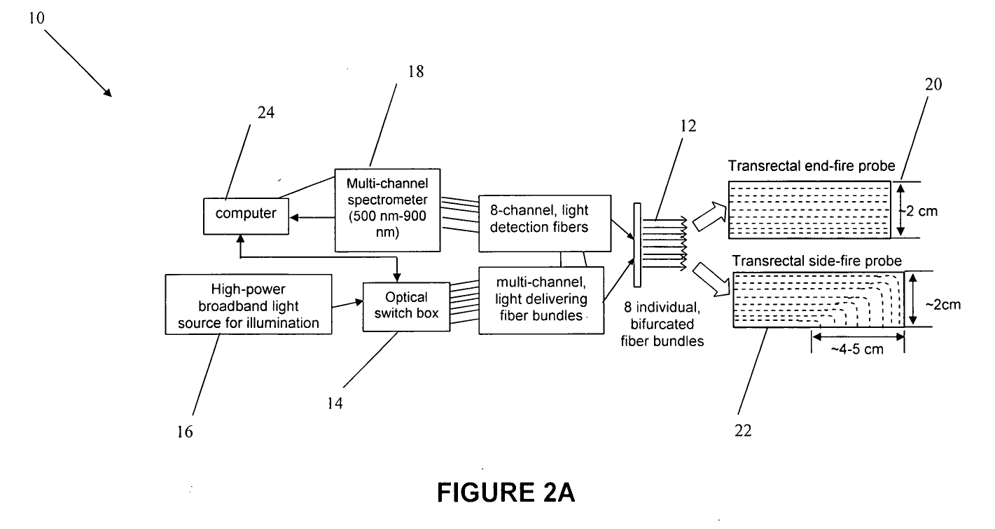

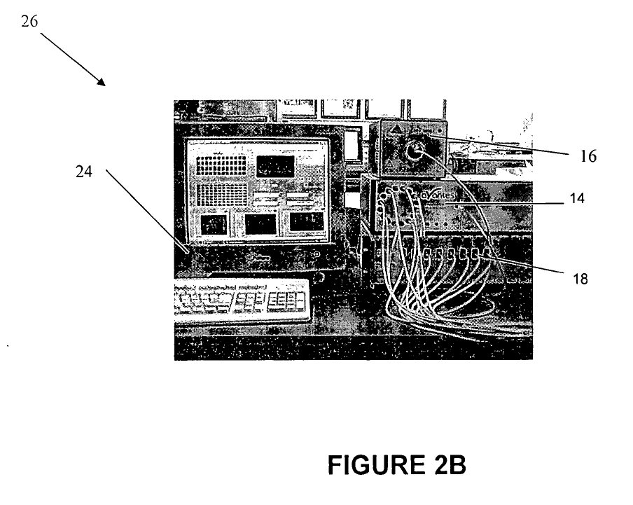

For the measurements of simulated or human prostate gland, a preferred embodiment of the present invention may use a CW, broadband light source such as Tungsten Halogen. A CW, broadband light source provides several advantages, including: 1) a broadband spectrum, allowing to obtain more accurate calculations in Hb, HbO, and light scattering parameters; 2) the CW imager results in the largest penetration depth and best signal-to-noise ratio in comparison to TD and FD systems; and 3) the optical signals from a white light source are strong enough to be detected through 3-4 cm thickness of a simulated or real prostate gland. Thus, a preferred embodiment of the present invention comprises multi-bifurcated fiber bundles with 4-5 mm in diameter for both light delivery and collection. The present invention integrates these multi-fiber bundles into transrectal probes. For example, FIG. 2A schematically diagrams a multi-source, 8-detector transrectal NIR imaging system 10 of the present invention and uses for example: (1) 8 bifurcated fiber bundles 12; (2) an 1-to-8 optical switch box 14; (3) a high power, broad-band light source such as a broad-band CW light source 16; and (4) a multi-channel spectrometer 18, such as an 8-channel spectrometer with CCD arrays with wavelengths of 500-900 nm.

FIG. 2A also depicts some example designs for the end-fire probes 20 and side-fire probes 22 (generally probes 23) in accordance with the present invention. A computer 24 drives the optical switch box 14, letting only one light-delivery channel open at one time. The computer 24 then performs data acquisition from the 8-channel detection fiber bundles 12 through the multi-channel spectrometer 18 and finally records and processes the data. The present invention thus advantageously limits the temporal resolution between two image acquisitions within a few seconds by either increasing light intensity and fiber diameters or using a faster optical switch box 14. A sample experimental setup is depicted in FIG. 2B with, for example, a computer 24, light source 16, optical switch 14 and multi-channel spectrometer 18.

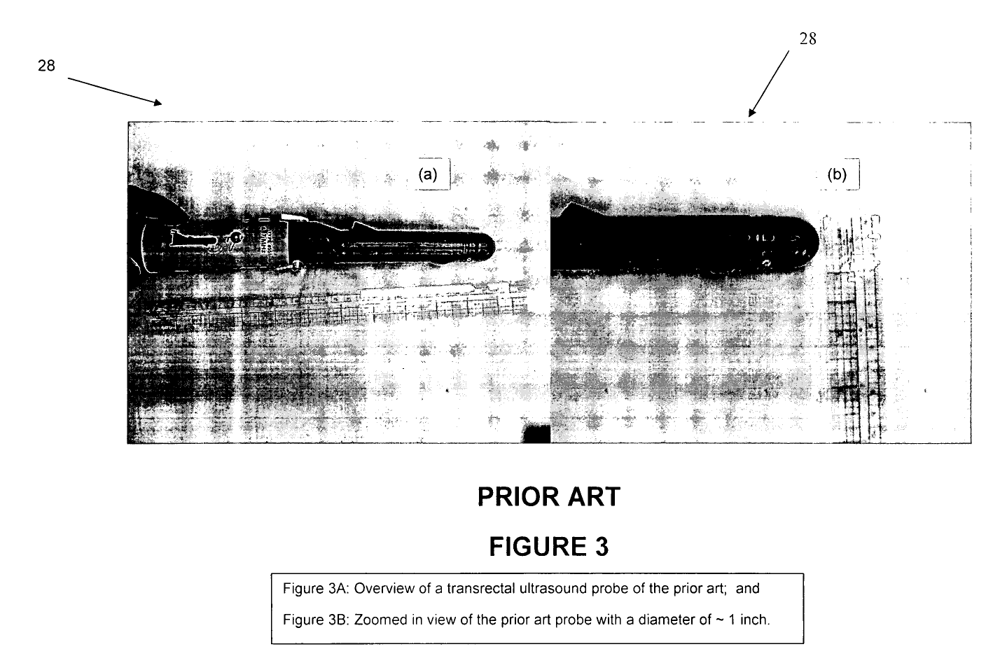

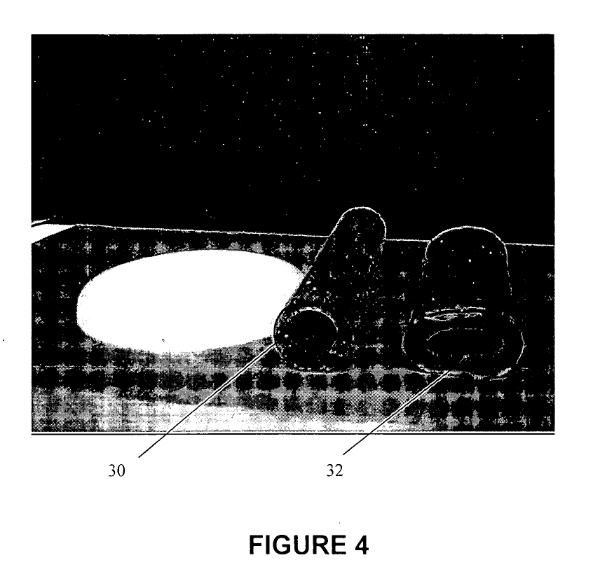

The design and implementation of the transrectal, NIR spectroscopic (end-fire probe 20) and imaging (side-fire probe 22) probes 23 in accordance with the present invention is based on the transrectal ultrasound probes 28 of the prior art and currently utilized in clinics, as illustrated in FIGS. 3A and 3B. However, the present invention comprises a rubber-like silicone to be the outer material 30 for the probe 23 for added comfort to the human rectum (see FIG. 4). A preferred outer material 30 comprises two translucent components mixed in a ratio of 10:1 by weight and exhibits a low viscosity and needs additional curing at room temperature. By using plastic silicone, for example, the probe shape may be designed through the mold. In addition, intralipid and ink can be added into the base material, before mixing with the catalyst, to introduce the light scattering and absorption. FIG. 4 also illustrates that a circular silicone holder 30 may be used for an end-fire probe 20 and the oval-shaped silicone holder 32 for the side-fire probe 22.

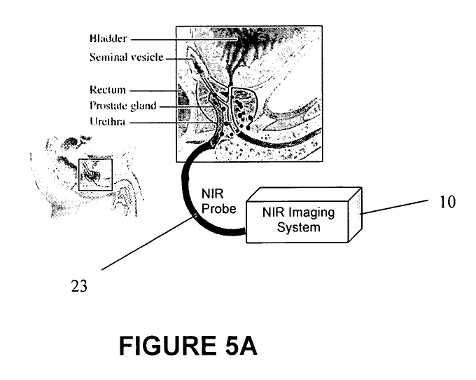

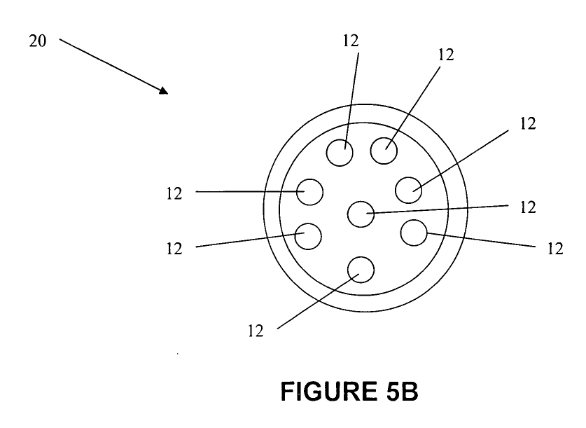

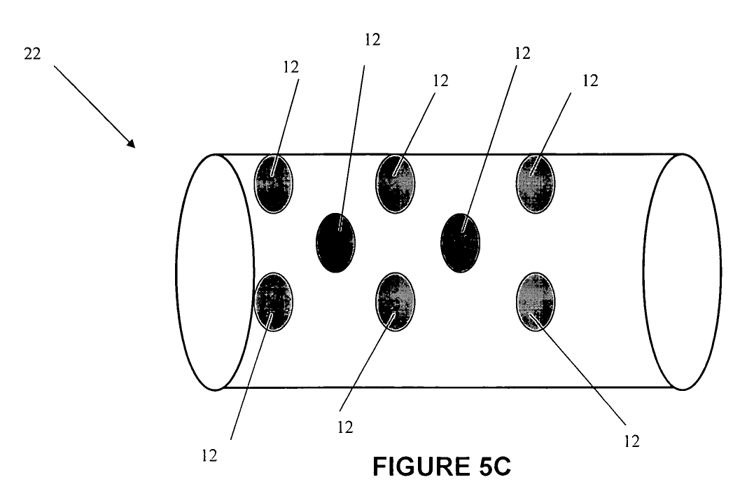

FIG. 5A illustrates the basic geometry of the transrectal probe 23 of the present invention including a curvature to match the curvature of human rectum. The probe head should be, for example, about ˜2 cm in diameter and made with a rubber cover 30, and the length should be about 8 cm. For an end-fire, transrectal NIR probe 20, the 8 bifurcated fiber bundle 12 shown in FIG. 5B, the NIR light can be delivered through one of the 8 fiber bundles and the rest of the bundles can detect the NIR signals. In addition, for a side-fire probe 22 in accordance with the present invention (FIG. 5C), multiple light sources and detector fiber bundles 12 are placed, for example, on the side of the oval-shaped probe so that the light can be delivered to and detected from deeper areas of the prostate gland. FIG. 5C illustrates an 8×8 channel side-fire probe 22, where all 8 bifurcated fiber bundles 12 may deliver and detect the NIR light, permitting 7×8 readings. The separation between the close source and detector may be designed such that it is approximately 0.5 cm.

Calibration for the Transrectal, NIR Tomographic Probes and Imagine System

Direct readings from the multi-channel imaging system 10 need to be calibrated to obtain equivalent readings that can be predicted by diffusion theory. A measured optical spectral intensity, Rₜᵢₛₛᵤₑ(λ, ρₛ, ρ_{d}), with a particular source/detector pair at ρₛ and ρ_{d} should be background subtracted and normalized to a standard calibration sample. Mathematically, it is expressed as:

$${R}_{cal} = \frac{{R}_{tissue} ( λ , {ρ}_{s} , {ρ}_{d} ) - {R}_{back\_tissue} ( λ , {ρ}_{s} , {ρ}_{d} )}{{R}_{standard} ( λ , {ρ}_{s} , {ρ}_{d} ) - {R}_{back\_standard} ( λ , {ρ}_{s} , {ρ}_{d} )} \qquad ( 10 )$$

where R_{back}_{_{—}}ₜᵢₛₛᵤₑ(λ, ρₛ, ρ_{d}) and R_{back}_{_{—}}_{standard}(λ, ρₛ, ρ_{d}) are the measured spectral intensities of the corresponding background with a particular source/detector pair from both the tissue and standard sample, respectively, and R_{standard}(λ, ρₛ, ρ_{d}) is the spectral intensity from the standard calibration sample with a particular source/detector pair at ρₛ and ρ_{d}. The standard sample is available in our lab with a high reflectivity of >99.9% and with a flat spectral band within 500 nm-900 nm. This calibration procedure needs to be conducted for each of the 8 light-delivery channels and 8 light-detection channels. Then, 7×8 calibrated readings of R_{cal} (λ, ρₛ, ρ_{d}) will be ready to use for the imaging reconstruction based on diffusion theory.

Multi-channel system 10 may be tested using the CW analytical solutions of the diffusion equation aids in quantifying absorption and reduced scattering coefficients. Using blood mixtures with intralipid; rabbit blood is collected and mixed with heparin to reduce clotting. One liter of a 0.5%-1% intralipid solution was made so that tlhe analytical diffusion solutions can be applied. Multiples of 10 ml of the blood were added into the solution and mixed thoroughly. A co-oximeter measures Hb, HbO, HbT concentrations, and hemoglobin oxygen saturation (SO₂) of the blood before the blood were added into the solution. To deoxygenate the blood mixture, a non-oxygen gas (such as N₂) were bubbled in the solution. Thus, bubbling pure O₂ gas made the solution oxygenated again. To calibrate the multi-channel NIR readings, the present invention is used as a reference to obtain accurate readings of SO₂. The optical source/detector filers are placed on the side of the liquid phantom container and measurements were taken in several oxygenating-deoxygenating cycles.

In order to obtain vascular Hb, HbO, HbT, and light scattering parameters, diffusion theory and spectroscopic approach were combined to analyze the steady-state diffuse reflectance, R, where R is the diffuse photon flux escaping from the tissue/boundary interface (i.e., at z=0). The reflectance can be measured through our NIR multi-spectral fiber bundles and is written as ‘R’ in Equation (11):

$$R ( ρ , {z}_{0} ) = \frac{{I}_{0}}{4 π} [ {z}_{0} ( {μ}_{eff} + \frac{1}{{r}_{1}} ) \frac{exp ( - {μ}_{eff} {r}_{1} )}{{r}_{1}^{2}} + ( {z}_{0} + 4 AD ) \times ( {μ}_{eff} + \frac{1}{{r}_{2}} ) \frac{exp ( - {μ}_{eff} {r}_{2} )}{{r}_{2}^{2}} ] \qquad ( 11 )$$

where

$${r}_{1} = \sqrt{{z}_{0}^{2} + {ρ}^{2}} , \\ {r}_{2} = \sqrt{{( {z}_{0} + 4 AD )}^{2} + {ρ}^{2}} , \\ {μ}_{eff} = \sqrt{\frac{{μ}_{a}}{D}} ,$$

I₀ is an overall amplitude factor, D is the diffusion coefficient and equal to

$$D = \frac{1}{3 \times ( {μ}_{a} + {μ}_{s}^{′} )} ,$$

z₀ is the depth of the isotropic point source of a pencil beam and equal to

$${z}_{0} = \frac{1}{{μ}_{t}^{′}} = \frac{1}{{μ}_{a} + {μ}_{s}^{′}} ,$$

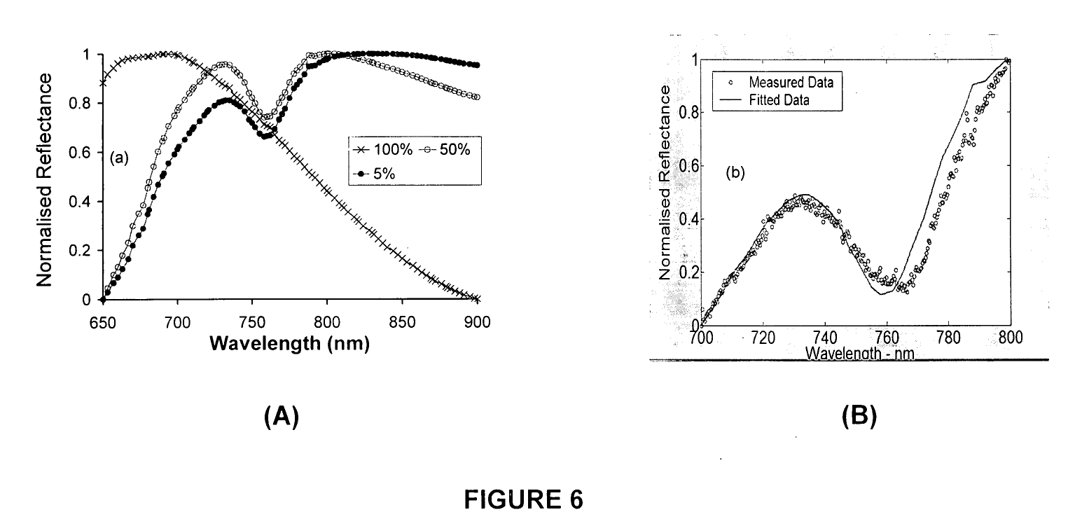

and A is a constant related to the internal reflection. In the diffusion regime, (i.e., when μa<<μs″ in the NIR region), Equation (11) has been shown in good consistency with spatially resolved Monte Carlo simulations with p larger than ˜1 mm. An improved solution is also available for more accurate characteristics of tissue optical properties for ρ>1 mm. Since the spectral dependence of absorption (μa) for blood-perfused tissues can be written as Equation (12):
μa(λ)=HbO*εHbO(λ)+Hb*εHb(λ) (12)
where λ is wavelength in nm, HbO and Hb represent concentrations of oxygenated and deoxygenated hemoglobin, respectively, and εHbO(λ) and εHb(λ) are extinction coefficients for HbO and Hb at λ, respectively. Moreover, because spectral dependence of light scattering (μs′) of tissue is weak it can be approximated as Equation (13):
μₛ′(λ)=aₛλ^{-p}^{ₛ} (13)

where as and ps are light scattering amplitude and power. By substituting Equations (12) and (13) into Equation (11), we can obtain a quantitative relationship between the parameters of Hb, HbO, as, ps and the measured light reflectance from the NIR multi-spectral fiber bundles in the wavelength range of 650 nm to 900 nm. This set of parameters (i.e., Hb, HbO, as, ps) can be obtained by fitting the equation with the experimental data, resulting in the final quantification of HbO, Hb, HbT, hemoglobin oxygen saturation, SO₂, light scattering amplitude, as, and scattering power, ps. FIG. 6A is plot of Equation (11) with different SO₂ levels (5%, 50% and 100%) and a source-detector separation of 2 cm, demonstrating that the measured NIR reflectance from 650 nm to 900 nm is highly oxygenation-dependent. FIG. 6B shows a possible fitting between the experimental and theoretical data in the wavelength range of 700 nm-800 nm. FIG. 6A illustrates the theoretical plots of normalized reflectance as a function of hemoglobin oxygen saturation (SO₂=100%, 50%, 5%). FIG. 6B illustrates a comparison of normalized reflectance obtained from the experiment data and theoretical calculation, using Equations (11), (12) and (13).

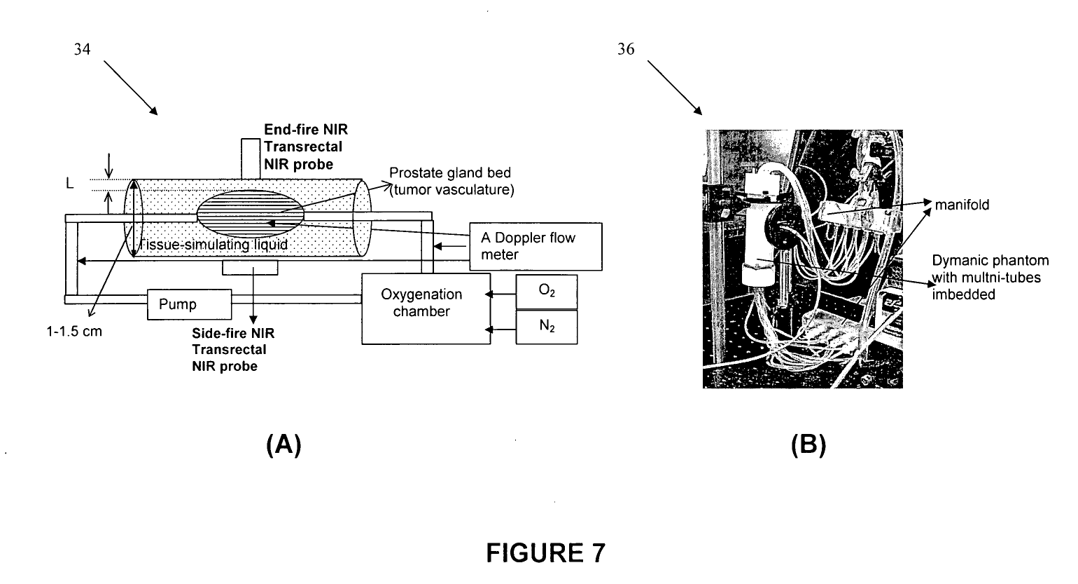

An experimental hemodynamic phantom to simulate the hemodynamic process in a prostate gland was built. FIG. 7A illustrates the arrangement and experimental setup for the hemodynamic phantom experiment 34, whereas FIG. 7B illustrates the actual setup 36. The tumor vasculature is made of plastic capillary tubing and connected to a larger circulation tube, which is subject to an oxygenation chamber. Animal blood was used in the circulating system and pumped by a mechanical pump with a flow speed control. The tissue-simulating liquid surrounding the simulated prostate tumor consisted of a mixture of animal blood and intralipid, which has optical properties similar to real tissue capillary bed. The deoxygenated blood was re-oxygenated in the oxygenation chamber and re-circulate back into the system. Thus, this hemodynamic phantom 34 can simulate the hemodynamic process with controlled parameters of flow by controlling the pumping rate. The transrectal NIR probes 23, either end-fire 20 or side-fire probes 22, will be placed in transmittance geometry along the circumference of the phantom tube with a distance to the simulated prostate gland of ˜5 mm (i.e., L=5 mm). With such a dynamic phantom, it was examined if the transrectal NIR probes 23 were able to detect Hb, HbT, dynamic response time (τ) and light scattering changes if the vascular flow was altered.

Forward Model Calculations with Simulated Tumor Tissue

Numerically, the diffusion theory, given in Equation (14), aids in calculating the distributions of the photon fluence rate inside tissue.

$$\frac{\partial }{\partial x} ( D \frac{\partial Φ}{\partial x} ) + \frac{\partial }{\partial y} ( D \frac{\partial Φ}{\partial y} ) - {μ}_{a} Φ + S = 0 \qquad ( 14 )$$

Where: D=1/(3 μs′), μa and μs′ are absorption and reduced scattering coefficients in the tissue, respectively, and Φ is the photon fluence rate in the tissue. The measured optical intensities, R, and Φ are related by R=|∇Φ|. Specifically, the Finite Element Method code aids in this calculation. In such a way, both temporal and spatial images of the solutions after the boundary conditions and input property parameters (i.e., μa and μs′) are entered into the program. Notice that μa and μs′ should be a function of position, r, and the measured data obtained from the NIR multi-channel spectrometer 18 are multi-spectral, from 500 nm to 900 nm. Then, Equations (12) and (13) can be rewritten as:
μa(r,λ)=HbO(r)*εHbO(λ)+Hb(r)*εHb(λ), and μₛ′(r,λ)=aₛ(r)λ^{-p}^{ₛ}^{(r)} (15)

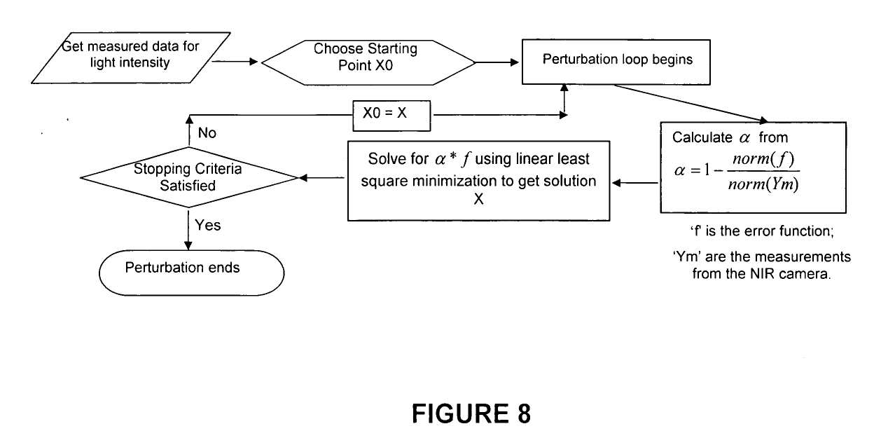

Accordingly, both R(r) and Φ(r) will be functions of HbO(r), Hb(r), as(r), and ps(r) at a particular position, r. The present invention thus provides an effective optimization approach to reconstruct tomographic images of Hb, HbO, HbT, and light scattering power and amplitude based on the NIR imaging measurements. The present invention finds the distribution of properties (i.e., photon fluence rates) to best fit the measured data. The image reconstruction is achieved by Sequential Quadratic Programming with Gauss-Newton Algorithm (SQPGNA), shown in the flow chart depicted in FIG. 8. In SQPGNA, the model parameters (i.e., absorption and scattering coefficients) are adjusted gradually to minimize the error between the predicted and measured outputs. Instead of traditional practice of correcting the entire error function in one step, the algorithm corrects a small fraction of error function. In each step, a constrained (with bounds) linear least square problem is solved to minimize the error for that step. The solution obtained thus serves as the starting point for next step with different fraction of error function. The magnitude of the fraction of error function is norm of error function normalized with the measurements, with maximum 20% increase allowed from the previous step.

The multi-spectral approach of the present invention provides NIR readings from 500 nm to 900 nm, having several hundred data points available for the imaging reconstruction. Since R(r) and Φ(r) are functions of HbO(r), Hb(r), as(r), and ks(r) at a particular position, r, the present invention collectively optimizes HbO, Hb, as, and ps by combining Equation (15) and SQPGNA. The final optimized HbO(r), Hb(r), as(r), and ks(r) are the desired reconstruction images of Hb, HbO, and the light scattering amplitude and power, i.e., as(r) and ks(r). In this way, the present invention uniquely separates the scattering and absorption effects using the CW approach.

Thus, the present invention demonstrates that a transrectal, end-fire, NIR spectroscopic probe 20 with 1×8 channels for imaging human prostate in vivo may be implemented and tested with linearity and stability. The relative tolerances for non-linearity and non-stability are 10% or less. The present invention also demonstrates that multi-parameter readings of oxygenated hemoglobin concentration (HbO), deoxygenated hemoglobin concentration, total hemoglobin concentration (HbT), light scattering amplitude and power (aₛ and pₛ), and dynamic response time (τ) have been derived from the end-fire NIR spectroscopic probe 20 using laboratory phantoms.

The present invention further demonstrates that a transrectal, side-fire, NIR tomographic imaging probe 22 with 7×8 channels for imaging human prostate in vivo has been implemented and tested with their linearity and stability. In addition, the present invention demonstrates that the tomographic imaging reconstruction algorithms have been developed and validated for transrectal, prostate images of HbO, Hb, HbT, aₛ, pₛ, and τ, using laboratory phantoms, within a relative error ≦10%.

It is advantageous if prostate cancer can be screened quantitatively and accurately before needle biopsy takes place. Thus, by combining the NIR tomographic imaging technique with the novel design of transrectal NIR probes 23, the present invention obtains physiological signatures of prostate cancer in humans, which can be, in turn, used later as finger-prints of prostate cancer to diagnose the cancer. Through this development, the tomographic images of values/changes in HbO, HbT, light scattering pattern, and hemodynamic response time from human prostate can be obtained minimally invasively. Such images are physiologically meaningful, providing diagnostic information on inhomogeneous and abnormal development in the human prostate vasculaure. The outcome present invention provides imaging modality to improve/enhance accuracy of prostate cancer screening as well as to significantly decrease the biopsy rate.

The near infrared imaging probe 23 of the present invention may be used in vivo transrectally for minimally invasive diagnosis of prostate cancer in human. If this hypothesis is proven true from the proposed study, such a portable, cost-effective, screening tool can be utilized in routine check-up clinics in the near future. The NIR transrectal imaging may also have a potential to be combined with transrectal ultrasound for better functional imaging in the future.

Two NIR transrectal probes 23 with 1×8 and 8×8 channels have been implemented and tested for their linearity and stability. In addition, NIR tomographic imaging of HbO, Hb, HbT, aₛ, pₛ, and τ from human prostate has been achieved using the laboratory dynamic phantoms. According, the NIR transrectal imaging probe 23 of the present invention may be used in vivo for minimally invasive diagnosis of prostate cancer in human. The clinical data obtained by using the end-fire spectroscopic probe characterizes the physiological signatures of normal prostate vasculature versus the same for prostate cancer. NIR tomographic imaging in accordance with the present invention utilizes multi-parameters (HbO, Hb, HbT, aₛ, pₛ, and τ) from the human prostate and provides the screening and diagnostic values of those parameters for prostate cancer.

The present invention provides tomographic images of values/changes in Hb, HbO, light scattering amplitude and power, and hemodynamic response time from human prostate can be used to identify physiological signatures of the human prostate cancer, and thus serves as prostate cancer screening and diagnosis tools for use in, for example, daily clinical practice. The present invention provides, for example, an 1×8 NIR, end-file, spectroscopic probe 20 to obtain clinical data from both potential prostate cancer patients and control subjects, and further to quantify the values of HbO, HbT, light scattering amplitude and power, aₛ and pₛ, and hemodynamic response time, τ, for each case. The present invention further provides identifiers for prostate cancer signatures using HbO, Hb, HbT, aₛ, pₛ, and τ, as well as the spectral features of the NIR data, by comparing with biopsy results from the corresponding subjects.

In addition, the present invention uses an 8×8 NIR side-fire tomographic imaging probe 22, to obtain more clinical data for tomographic images of HbO, Hb, HbT, aₛ, pₛ, and τ from potential prostate cancer patients. The present invention provides a screening/diagnostic tool for prostate cancer; the tomographic images of HbO, Hb, HbT, aₛ, pₛ, and τ, as well as the spectral features of NIR signals, will be obtained and then compared to those with, for example, TRUS biopsies.

Although preferred embodiments of NIR tomographic imaging techniques and transrectal NIR imaging probes 23 for detecting human prostate cancer have been described in detail herein it will be appreciated that the present invention provides many applicable inventive concepts that can be embodied in a wide variety of specific contexts. For example, while the description has principally been used to detect human prostate cancer, it should be understood that the system may also be utilized for detecting other abnormalities and cancers. As another example, although the description includes an example of an 8 channel imaging system, systems in accordance with the present invention employing any number of channels may be used. The specific embodiments discussed herein are merely illustrative of specific ways to make and use the invention, and do not delimit the scope of the invention. Those skilled in the art will recognize that various substitutions and modifications may be made to the invention without departing from the scope and spirit of the appended claims.

What is claimed is:

1. A multi-channel, near infrared (NIR) imaging system comprising:
one or more optical fiber bundles; and
a transrectal NIR probe having an outer material, wherein the optical fiber bundle is integrated into the transrectal probe.

2. The multi-channel NIR imaging system of claim 1, wherein the transrectal NIR probe is an end-fire NIR probe.

3. The multi-channel NIR imaging system of claim 1, wherein the transrectal NIR probe is a side-fire NIR probe.

4. The multi-channel NIR imaging system of claim 1, wherein the outer material is rubber-like silicon.

5. The multi-channel NIR imaging system of claim 1, wherein outer material is made of two translucent materials exhibiting low viscosity and requiring curing at room temperature.

6. The multi-channel NIR imaging system of claim 1, wherein the transrectal NIR probe comprises a curvature to match the curvature of a human rectum.

7. The multi-channel NIR imaging system of claim 1, wherein the outer material is a circular silicone holder.

8. The multi-channel NIR imaging system of claim 1, wherein the outer material is a oval-shaped silicone holder.

9. The multi-channel NIR imaging system of claim 1, further comprising a broad-band light source.

10. The multi-channel NIR imaging system of claim 9, wherein the broad-band light source is tungsten halogen.

11. A multi-channel, near infrared (NIR) imaging system comprising:
one or more optical fiber channels;
a transrectal NIR probe having an outer material, wherein the optical fiber channels are bundled into the transrectal probe;
an optical switch box adapted to allow signals from only one of the fiber channels to pass through at a time;
a broad-band light source adapted to deliver light to the fiber channels; and
a multi-channel spectrometer to capture data from the channels.

12. The multi-channel NIR imaging system of claim 11, wherein the NIR imaging system is adapted to capture tomographic images of changes in HbO, HbT, light scattering patterns and hemodynamic response times from a human prostate.

13. The multi-channel NIR imaging system of claim 11, wherein the optical switch box is an 1-to-8 optical switch box.

14. The multi-channel NIR imaging system of claim 11, wherein the multi-channel spectrometer is an 8-channel spectrometer with arrays of wavelengths between 500-900 nm.

15. The multi-channel NIR imaging system of claim 11, wherein the temporal resolution of the imaging system between two image acquisitions is within 5 to 10 seconds.

16. The multi-channel NIR imaging system of claim 11, wherein the transrectal NIR probe is an end-fire NIR probe.

17. The multi-channel NIR imaging system of claim 11, wherein the transrectal NIR probe is a side-fire NIR probe.

18. The multi-channel NIR imaging system of claim 11, wherein the outer material is rubber-like silicon.

19. The multi-channel NIR imaging system of claim 11, wherein outer material is made of two translucent materials exhibiting low viscosity and requires curing at room temperature.

20. The multi-channel NIR imaging system of claim 11, wherein the transrectal NIR probe comprises a curvature to match the curvature of a human rectum.

21. The multi-channel NIR imaging system of claim 11, wherein the outer material is a circular silicone holder.

22. The multi-channel NIR imaging system of claim 11, wherein the outer material is a oval-shaped silicone holder.

23. The multi-channel NIR imaging system of claim 11, wherein the broad-band light source is tungsten halogen.
